(19) United States
(12) Patent Application Publication
(10) Pub. No.: US 2009/0005718 A1
(43) Pub. Date: Jan. 1, 2009

(54) COMPRESSION, THERMAL AND SUPPORT BANDAGING SYSTEM

(75) Inventor: Warwick Douglas Lightbourne, Auckland (NZ)

Correspondence Address: YOUNG & THOMPSON, 209 Madison Street, Suite 500, ALEXANDRIA, VA, 22314, US

(73) Assignee: CLINICAL TECHNOLOGY LIMITED, AUCKLAND (NZ)

(21) Appl. No.: 11/769,208

(22) Filed: Jun. 27, 2007

(51) Int. Cl. A61L 15/00; A61F 5/00

(52) U.S. Cl. 602/75; 602/27; 602/5

(57) ABSTRACT

A re-usable bandaging system for treating acute soft-tissue injuries and the like provides protection, support and restraint, and optional heating or cooling and uses straps enabling the system to be placed at one of many sites. A flexible, resilient insulating material, covered with loop-bearing fabric comprises (A) a main bandage that is a rectangular panel with a pocket and (B) two straps at diagonally opposite corners. Each strap has an area of complementary hooks at the free end, to adhere to the bandage when in use. A supplementary bandage (C) is for optional use as a strap when stabilising the main bandage. The system provides a sealed flexible gel pack for heating or cooling an injured part when placed in the pocket. The pack is internally baffled to evenly apply a compress therapy by restriction motion of the contents.

### FIELD

This invention relates to the general field of therapeutic equipment for treating bruises, strains, other soft tissue injuries, and weak joints with particular application to means for applying support bandaging with the option of a hot or cold compression to an injured part of the body.

### BACKGROUND

Injuries sustained during sports and other physical pursuits often involve impact damage to muscle tissues, or strains and sprains resulting from over-stretching or over-turning of connective tissues around joints. With these types of injuries, it is often desirable to reduce swelling and pain in the short term by quickly applying ice or a cold compress to the injured area. Over the longer term, pain from such injuries as well as back, neck or joint pain may be relieved by the application of a hot compress to dilate blood vessels and improve blood flow through the area.

Furthermore, it is often useful to partially or completely immobilise or support the joint or joints associated with the damaged area, to reduce damage or to assist action of the muscles during recovery or rehabilitation.

### PROBLEM TO BE SOLVED

It is desirable for a compress to be kept available at sports events and the like, to treat these kinds of injury as quickly as possible. However, keeping ice and/or cold water available on site over an extended period may be difficult or inconvenient. Furthermore, the water and/or ice can be difficult to control and retain on the injured part as it melts and runs out of the compress, particularly if the injured part is elevated as is recommended to minimise swelling.

To be effective, the compress (whether cold or hot) should be strapped relatively firmly and securely on to the injured part of the body. By this means the injured part is not only treated with temperature regulation but also given support and pressure to reduce swelling, and is prevented from further movement or twisting. However, given the range of different sizes, shapes and orientations of body parts which might be injured, fastening a relatively heavy and fluid compress firmly on to a particular body part with a bandage, strap or tape can require considerable skill and experience. Specific straps or bandages can be used to suit application on a particular injury site such as the wrist or ankle, but it may be difficult or impractical to keep a full range of such specific straps on hand, particularly if by chance several people suffer injuries to the same part of the body. Generic prior-art straps intended to be applicable to any or various different parts of the body give only a compromised degree of support to the body part without providing acute injury immobilisation and restraint, or joint movement support, depending on the requirement.

### PRIOR ART

There are many publications within the broad area, such as Chalek U.S. Pat. No. 6,936,018 which is capable of holding a hot or cold compress in the middle of a long bandage for application to a body. It would not stay in place very well. Bergman U.S. Pat. No. 4,214,581 provides a triangle-folded bandage means capable of holding a hot or cold compress against a body. Plewes U.S. Pat. No. 6,656,210 provides an encircling band, but it would not reach joints such as the hip. Wendel US 2003/0194426, like Westplate U.S. Pat. No. 4,592,358 provides a multi-pouch package in various types. Hickling in U.S. Pat. No. 5,743,867 provides a strap-free wrapping device for limbs and ankles. Possibly most similar to the present invention is U.S. Pat. No. 6,589,272 Sheikh which includes straps and a central panel, but this invention appears to require a variety of different bandages to reach a variety of sites. Edwards et al in U.S. Pat. No. 6,440,159 attempts a more-or-less strap-free version. None of these seem to have the general versatility of the present invention, as described elsewhere in this specification.

### OBJECT

It is an object of this invention to go at least partway towards providing a tension adjustable strapping system for applying and retaining an optionally cold or hot compress on the injury site and/or at least partially supporting or immobilising the injured part or a weakened or degenerative limb; or at least to provide the public with a useful choice.

### STATEMENT OF INVENTION

In a first broad aspect the invention provides a re-usable compression, thermal and support bandaging system (the thermal aspect being optional), wherein the system includes a main bandage having an approximately central wider area having a breadth, a width, an outer and an inner surface; joined to two long flexible straps each having a free end and a joined end; each strap extending in opposite directions from diagonally opposite portions of the wider area; each strap and the outer side of the wider area being covered with a surface comprising a first part of a self-fastening means capable of forming reliable, reversible bonds with a second complementary self-fastening means applied about the free end surfaces of the straps so that no external fixing means is required when placing the bandage against an injured or diseased part of a subject.

Preferably the wider area is substantially square; alternatively the wider area is rectangular.

Preferably the two long flexible straps of unequal length each having a free end and a joined end.

Preferably all the straps are comprised of a flexible, elastic and resilient material. Preferably the straps, at least on an outer side, are formed from fabric-covered neoprene material. Preferably the fabric-covered neoprene material is washable so that the bandage system may be re-used.

Preferably the straps are permanently joined on to a central wider area having a breadth and a width, an outer surface and an inner surface; the straps extending from the wider area in opposite directions; each long strap and the wider area being comprised of a flexible, elastic and resilient fabric-covered neoprene material.

Alternatively the straps are permanently joined by their side edges over a distance up to the joined end, so that the double width of strap comprises the wider area.

Preferably the straps are at least 350 mm in length (sizes being adapted for a human subject of average adult height, although bandage systems of other sizes may be provided for children and for patients with degenerative or like pathologies, (and for animals or specific parts thereof, such as racehorses and dogs).

Preferably the straps are between 400 mm and 850 mm in length. Preferably the straps are at least 50 mm wide; more preferably between 80 mm and 150 mm wide. Optionally one strap is longer than the other strap; preferably about 25% longer and both are sufficiently long to pass at least once around an injured subject.

In one option, the straps are permanently joined to each other along part of an adjoining side towards an end of each strap thereby comprising the wider area; the straps extend in opposite directions from diagonally opposite corners of the wider area.

Preferably the bandaging system employs reversible contact adhesion such as provided by “Velcro®” branded material; for which the majority of the covering is a washable “eye” or complementary bondable material; and relatively small areas of reversibly adhesive hook material are attached to a free end surface of both flexible straps; the reversibly adhesive hook material being capable of forming a secure yet reversible bond on to any other part of the fabric-covered neoprene main bandage, thereby allowing the compression and support bandaging system to be wrapped in place on to a variety of sites on the body so that the inner surface is placed against an injured part of an injured subject.

In one option the “eye” material is provided on an outer surface of the bandage only: in another option the “eye” material is provided on both sides.

In another related aspect, the invention provides a re-usable compression, thermal and support bandaging system wherein the amount of compression to be applied to the injured part is predetermined by adjusting the tension in the resilient straps before making the bond.

In yet another aspect, the system includes at least one second bandage comprising a further elongate spica strap. A re-usable compression, thermal and support bandaging system as claimed in claim 1, wherein the system also provides a temporarily attachable supplementary bandage (herein termed a spica overstrap) comprised of an elongated strap of a flexible, elastic and resilient material covered with a fabric; the strap including at both ends a second complementary self-fastening means applied about the free end surfaces of the straps capable of bonding reversibly to the remainder of the spica overstrap or to the straps or central area of the main bandage, so that the support provided by the main bandage can be enhanced by attachment of the spica overstrap, thereby providing further support to injured parts and improving retention of the bandaging system on an injured person.

In one option, the support provided by the main bandage can be enhanced by attachment of the supplementary bandage, thereby providing further support to injured parts and improving retention of the bandaging system on an injured person.

Preferably the supplementary bandage comprises a spica overstrap at least 600 mm in length, for use as a stabilising spica overstrap.

In yet another related aspect, the wider area has a breadth and a width capable of enveloping a thermally active flexible bag within a pocket adjacent the inner surface so that an injured part of the subject may be effectively exposed to the bag so that it may be heated or cooled as required.

Preferably the pocket is formed between two layers of said carrier, an inner layer comprising an insulating fabric, and an outer layer comprising a durable neoprene material.

In a further related aspect, the thermally active flexible bag is sealable and is capable of holding an effective amount of either a pre-heated or a pre-cooled temperature-moderating mass so that, when in use, heat can be transferred to or from the injured part of the subject located beneath the wider area of the bandage; the bag including internal, partial or complete cross-bridges so that the mass is held in substantially even distribution within the bag despite a tendency of gravity or locally applied pressure to cause redistribution of the mass.

In a second broad aspect, the flexible container that may be placed inside the pocket is relatively flat and includes a plurality of internal, partial or complete interconnections between one side and the other, resembling a quilted arrangement thereby providing baffles, so that movement or pooling of the fluid mass within the container when in use is reduced.

Preferably the temperature-moderating fluid mass comprises a material having an effective specific heat of about 1 or greater.

In a third broad aspect the invention provides a compression and support bandaging system as previously described in this section, wherein the system is proportioned and dimensioned so as to be usable in situations selected from a range including (a) small joints of adult humans, (b) injured children (c) for injured animals or (d) for degenerating joints. Preferably, elderly humans and variously disabled humans are catered for.

In a fourth broad aspect the invention provides a method for using a compression and support bandaging system as previously described in this section over an identified injury which has been identified and for which appropriate treatment has been at last initially set, wherein the method includes the steps of:

1. Optionally loading a thermally active bag with hot or with cold contents, sealing the bag, and placing the bag inside the pocket of the wider part so that thermal treatment may be done

2. Placing the inner surface of the wider part (including the pocket) against the injured site while wrapping the straps around the injured person or animal, and fasting the strap ends against the supported surface so that the injured site is at least under a little compression and optionally also receiving thermal treatment; and

3. Optionally further stabilising the injured site and/or securing the compression and support bandage by placing the spica bandage as a further stabilising means so that the injured part is protected, compressed, supported and treated.

### PREFERRED EMBODIMENT

The description of a preferred form of the invention to be provided herein, with reference to the accompanying drawings, is given purely by way of example and is not to be taken in any way as limiting the scope or extent of the invention. Throughout this specification unless the text requires otherwise, the word “comprise” and variations such as “comprising” or “comprises” will be understood to imply the inclusion of a stated integer or step or group of integers or steps but not the exclusion of any other integer or step or group of integers or steps. Sizes provided herein are generally suited to use on a human subject of average adult height, although bandage systems may also be provided for children who of course also play sports or become injured (and for animals, such as racehorses and dogs.). Specific sizes as given in this specification are generally found suitable for use on nearly or actually fully-grown humans (having a height of about 1.8 metres or 6 feet).

### DRAWINGS

FIG. 1: shows a preferred flexible container, with a fluid mass of gel having high specific heat sealed into it to form a compress.

FIG. 2: shows a first bandage of the invention, suited for use with the compress of FIG. 1.

FIG. 3: shows the preferred compress applied with the carrier to (a) the back, (b) the ankle and (c) the thigh using the first bandage of FIG. 2.

FIG. 4: shows a second bandage of the invention, suited for use with the bandage of FIG. 2.

FIGS. 5(a) and 5(b): show the first and second bandages used together to bind an ankle.

FIG. 6: shows the first and second bandages used together to bind a hip.

There are several ways that this invention or system can be used. It can be used to hold a compress against a part of the body, thereby aiding in (a) reduction of inflammation, (b) recovery of function, (c) reduction of discomfort, and so on, according to general principles of medicine. The compress may be hot or cold, as required. It can simultaneously or separately be used to restrict movement, particularly with the help of the second, separate bandage. That is useful for example in the case of ankle, knee or hip joint, spica and stress-related injuries such as sprains, tendon stretch or rupture, and the like.

### EXAMPLE 1

The panel 22 of the bandage 20 is preferably about 200 mm (8 inches) wide and 270 mm (11 inches) long and made of a resilient material such as a closed-cell neoprene (perhaps laminated) with an outer surface-fastenable covering. The included straps 23 and 24 are preferably about 480 mm long, and extend in opposite parallel directions from opposing diagonal corners of the panel 22, as shown in FIG. 2. The straps need not necessarily be of equal length, and could be anything from 400 mm (16 inches to 850 mm (22 inches) in length. In particular, the combination of a significantly shorter strap with a significantly longer one might be used. This has been found to be advantageous when strapping a person. Both straps have a hook-bearing attachment pad 26 at the end, adapted to removably fasten on to the loop-bearing fabric of the outer surface 25. Because the entire outer surface 25 of the carrier 20 is comprised of the loop-bearing fabric 25, the straps 23 and 24 can be removably fastened on to any part of the bandage 20, including themselves, the panel 22 or the other strap. Use of the commercial product “Velcro®” or similar as the “hook and eye” type reversibly attachable surfaces, reversible adherent or fastening means has rendered the invention easy to apply and remove. The invention incorporates that commercial product and equivalents thereof.

The width of the straps is not considered to be critical, but it will be appreciated that wider straps will make the bandage more bulky and cumbersome, while very narrow straps may be weak and too stretchable, and/or tend to cut in on the body uncomfortably. A width range of between 80 mm and 150 mm is considered preferable at present.

This configuration of bandage can be strapped on to almost any part of the body, regardless of the size or proportions of the person, and can be used to bind and apply pressure and/or securely hold a compress such as the compress 10 or other dressing in place. As shown in FIG. 3(a), the bandage 20 can be strapped diagonally around the torso to press the compress 10 on to the back, shoulder, hip, chest or stomach, by stretching the straps 23 and 24 around the body and fastening them to each other at whatever length is necessary to firmly hold the compress 10 in place. As shown in FIG. 3(b), the compress can also be applied to an ankle, elbow, knee or wrist, with the strap 23 winding around the limb above the injured joint and fastening back on to itself, and the strap 24 winding around and fastening to itself below the joint, substantially at right angles to the strap 23. The compress 10 conforms to the shape of the joint, applying pressure and helping to immobilise it, as well as heating or cooling it. As shown in FIG. 3(c), the carrier 20 can also be fastened on to the thigh, calf, upper or lower arm with the two straps 23 and 24 winding back on to themselves in a substantially parallel configuration. For removal, the strap is simply pulled straight off the fabric.

In the case of an injured or weak joint such as an elbow, shoulder, ankle or hip it is desirable to immobilise the joint as much as possible, to control movement which can both be painful and also aggravate the injury. For this purpose a second separate bandage 30 is provided (herein termed a spica bandage), by which additional pressure and support can be applied around the joint to hold it in place. The use of these two bandages in combination allows the bandaging to easily be specifically tailored to the type and site of the injury, and the body size and shape of the injured person.

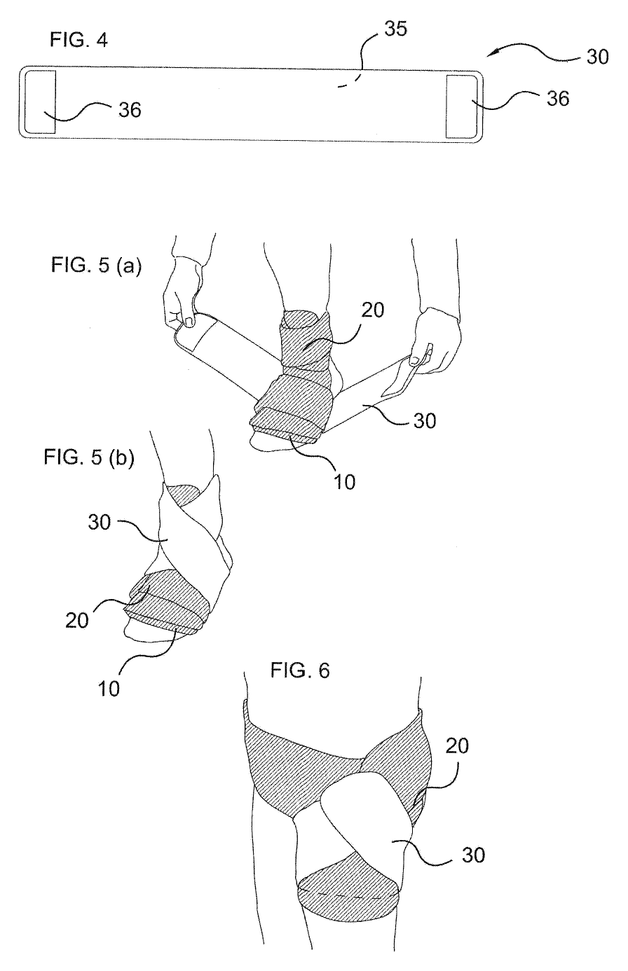

As shown in FIG. 4, the second bandage 30 comprises a straight spica strap, preferably about 620 mm (16 inches) long by 100 mm (4 inches) wide, formed of fabric-covered neoprene like the first bandage 20. The second bandage 30 has a hook-bearing attachment pad 36 at each end, adapted to removably fasten on to the loop-bearing fabric of the outer surface 25 of the bandage 20, and also on to the corresponding outer surface fabric 35 on the second bandage itself.

As shown in FIG. 5, the second bandage 30 can be applied over the bandage 20 (shown shaded) when binding an ankle injury as shown in FIG. 3(b). The bandage 30 is drawn under the instep of the foot as shown in FIG. 5(a) and crossed over the top of the foot and ankle to bind around the lower calf, attaching either to itself (the pad 36 at one end attaching to the back of the strap at the other end) or to the loop-bearing surface of the underlying first bandage 20. By this means the ankle support provided by the first bandage 20 is effectively and easily reinforced with the spica overstrap bandage 30.

Similarly, the second bandage can be applied together with the first to help immobilise or support an injured or degenerative hip as shown in FIG. 6. In this case, the first bandage 20 (with or without the use of a compress 10) is applied across the hip with the panel 22 on the outer hip, one strap 23 as a belt around both hips below the waist and the other 24 around the upper thigh of the leg. The second bandage 30 is applied over this as shown, passing around the inner upper thigh and up diagonally across the outer hip in the form of a hip spica support. This feels more secure and further reassures the patient.

### EXAMPLE 2

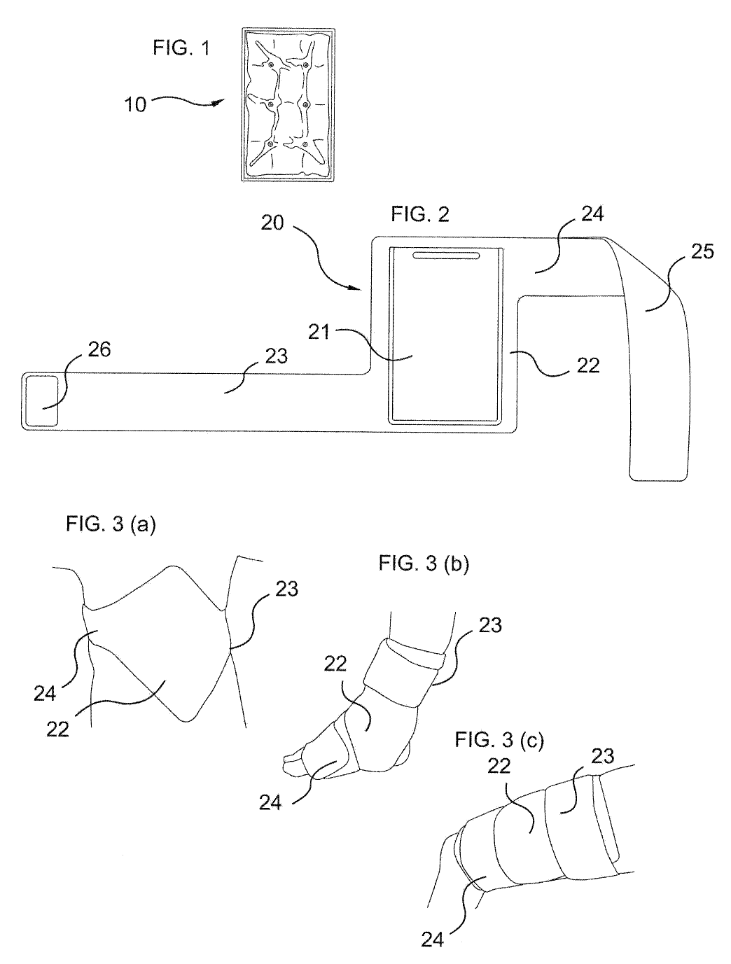

The preferred embodiment of the invention may be used with a compress 10 as shown in FIG. 1, held in a pocket within the wider area in the middle of the main bandage. The compress employs a mass of fluid gel sealed into a flexible container to apply a temperature. The gel is intended to retain a particular temperature—whether hot or cold—for an extended period, and should therefore comprise a relatively weighty mass. It should also be relatively fluid, so that it can conform to the shape of the body part to which it is applied and enable heat transfer across a wide area of the body surface. The mass may include materials exhibiting a latent heat-related state change. Variants may include or comprise electrically or chemically powered heating means.

The container may be formed from two sheets of a flexible and tough plastics material such as a vinyl, polypropylene or polyethylene, having a generally rectangular shape and heat-welded together around the edges (12) to provide a reliably sealed envelope. In addition to the welds around the edges, the two sheets are also welded together at an array of points across the face of the container, to quilt the container and bonding the interior surfaces to each other but allowing space for the fluid in between. These quilting points 11 hold the container in a flat pad-like configuration, and also act as baffles to inhibit gravity or pressure-driven flow of the fluid gel inside the container. One problem that prior-art containers have is that the gel within may flow under gravity and collect at the lowest point. Or, compression, perhaps caused by uneven bandaging or by subsequent swelling of the injured part drives the gel away from the site where it is most needed. In the preferred form of the invention, the container forms a pad about 150 mm (6 inches)×240 mm (11 inches), and has an array of six weld points (11), spaced about 60 or 70 mm (2.5-3 inches) apart. The gel is able to flow out around hard points such as a knee, knuckle or other joint, and into dimples and hollows, and thereby conform closely to the shape of the body part it is pressed on to.

The compress 10 is fastened on to the body with the use of a bandage 20. The bandage 20 preferably includes a pocket 21 shaped to hold the compress 10, although a simple panel without a pocket could also be used either to strap such a compress or a wound dressing on to the body part, or simply to strap, support and insulate the body part. In either case, the bandage 20 preferably has a generally rectangular panel 22 and two straps 23 and 24. The bandage 20 is largely formed from neoprene or a similar slightly elastic and flexible insulating material, and has an outer surface 25 covered with a loop-bearing fabric. If provided with a pocket 21, it is preferably formed from a porous, relatively light fabric sewn on to the neoprene body 22 of the carrier 20, so that heat transfer can easily occur through the light fabric of the pocket 21 between the compress 10 and the body part to which it is applied. The insulative material of the body 22 will however inhibit heat transfer from the compress 10 out to the environment, helping to keep it cool or warm as the case may be.

### Variations

While the embodiment described above is currently preferred for simplicity and ease of use and of re-use, it will be appreciated that many other variations might also be made under the general coverage of the invention. Two inclusive variations are the use of a medicated or impregnated pack within the panel 22, and also substitution of the compress panel 22 by a thermal therapeutic material lining.

Heat might be supplied by an electrical heating means within the pocket, or even by a chemical reaction. Of course, the internal material or gel may include a material (such as a wax) that undergoes a change of state from liquid to solid at a useful temperature (such as between 30 and 45 deg C.) and supplies heat; or changes from solid to liquid at about 5-15 deg C. and removes heat from the underlying body parts. Ice is an obvious material to apply over acute sprain-type injuries. More bags for the system as sold might be kept in a refrigerator or in an oven in order to have a supply of spares.

The inner surface of either or both bandages could also be provided with a loop-bearing fabric like the outer surface, so that the attachment pads at the ends of the straps can stick to any surface of either bandage. While the bandages will generally be applied in such a way that the pads contact only the outer surfaces, this would extend the range of options available for use of the bandages. In such a case the pocket 21 could be provided as a removable bag with its own attachment pads, or could be replaced with attachment pads provided directly on the compress 10, affixable on to either bandage at any point.

The shape and arrangement of the bandage 20 and the bandage 30 as shown is currently preferred, but it will be appreciated that some modifications might be made within the general scope of the invention. In particular the dimensions could be modified to provide (for example) a version applicable specifically to children. The rectangular shape of the compress 10 and hence the pocket 21 and the panel 22 of the bandage 20 might also be altered to a square, oval or other shape, without affecting the principles of the invention. Appropriate modifications in shape and size will provide optimised forms of the invention for veterinary applications or (if scaled down from the adult sizes described here) for use with children, who often engage in team or contact sports. Other sizes may be more suited to animal applications, such as for use on racehorse limbs for treating tendon and/or muscle and/or joint/bone problems; even laminitis. Apart from general use with pet animals, greyhounds used for racing may benefit from use of the invention.

The invention may be used in cases of (for example) joint degeneration (such as an arthritic hip), recovery after stress pathology affecting the musculoskeletal system, or myological or osteological disease being treated in a hospital or under extended care. The invention is in no way limited to use at a sports field. Size and shape alterations may be made according to specific purposes. For example, the bandage central area and the straps may be proportioned and dimensioned so as to be usable for a variety of purposes selected from a range including (a) injury control in small joints of adult humans, (b) injury control in children (c) injury control or stress remediation in animals or (d) management of degenerative conditions in one or more joints of humans. Elderly or disabled humans are included. Disabled persons including those with neurological problems are especially likely to require supportive bandages that are easy to put on and take off.

Finally, it will be understood that the scope of this invention as described by way of example and/or illustrated herein is not limited to the specified embodiments. Where in the foregoing description, reference has been made to specific components or integers of the invention having known equivalents, then such equivalents are included as if individually set forth. Those of skill will appreciate that various modifications, additions, known equivalents, and substitutions are possible without departing from the scope and spirit of the invention as set forth in the following claims.

I claim:

1. A compression, thermal and support bandaging system, wherein the system includes a main bandage having an approximately central wider area having a breadth, a width, an outer surface and an inner surface; joined to two long flexible straps each having a free end and a joined end; each strap extending in opposite directions from diagonally opposite portions of the wider area; each strap and the wider area being comprised of a flexible, elastic and resilient material covered on the outer side with a fabric comprising a first part of a self-fastening means capable of forming reliable, reversible bonds with a second complementary self-fastening means applied about the free end surfaces of the straps so that no external fixing means is required when placing the bandage against an injured or diseased part of a subject.

2. A compression, thermal and support bandaging system as claimed in claim 1, wherein the wider area includes bag retention means (a pocket) capable of enveloping a thermally active flexible bag adjacent the inner surface so that an injured or otherwise affected part of the subject is effectively exposed to the bag so that the part may be heated or cooled as required.

3. A compression, thermal and support bandaging system as claimed in claim 1, wherein the amount of compression to be applied to the part is predetermined by adjusting the tension in the resilient straps before making the bond.

4. A compression, thermal and support bandaging system as claimed in claim 2, wherein the thermally active flexible bag is sealable and is capable of holding an effective amount of either a pre-heated or a pre-cooled temperature-moderating mass so that, when in use, heat can be transferred between the part of the subject located beneath the wider area of the bandage and the bag; the bag including partial or complete internal cross-bridges so that the mass is held in substantially even distribution within the bag despite a tendency of gravity or locally applied variations of pressure to cause redistribution of the mass.

5. A compression, thermal and support bandaging system as claimed in claim 1, wherein the flexible straps are of unequal length, and are sufficiently long to pass around a bandaged subject.

6. A compression, thermal and support bandaging system as claimed in claim 5, wherein one flexible strap is about 25% longer than the other flexible strap.

7. A compression, thermal and support bandaging system as claimed in claim 1, wherein the system further includes a temporarily attachable supplementary bandage (herein termed a spica overstrap) comprised of an elongated strap of a flexible, elastic and resilient material covered with a fabric; the strap including at both ends a second complementary self-fastening means applied about the free end surfaces of the straps capable of bonding reversibly to the remainder of the spica overstrap or to the straps or central area of the main bandage, so that the support provided by the main bandage can be enhanced by attachment of the spica overstrap, use of the second bandage providing further pressure and support to at least one part of the subject and improving retention of the bandaging system at almost any site over the subject.

8. A compression and support bandaging system as claimed in claim 1, wherein the straps are permanently joined to each other along part of an adjoining side of each strap thereby comprising the wider area; the straps extend in opposite directions from diagonally opposite corners of the wider area.

9. A compression and support bandaging system as claimed in claim 8, wherein the system is proportioned and dimensioned so as to be usable for purposes selected from a range of subjects including (a) adult humans; including small joints such as fingers and wrists, (b) young adults, (c) children (d) elderly or disabled humans and (e) animals.
